(19) United States
(12) Patent Application Publication
(10) Pub. No.: US 2018/0055595 A1
(43) Pub. Date: Mar. 1, 2018

(54) MODULAR SHARPS CONTAINER SYSTEM

(71) Applicant: Sterilis, LLC, Boxborough, MA (US)

(72) Inventor: Gordon S. Lewis, Needham, MA (US)

(21) Appl. No.: 15/283,676

(22) Filed: Oct. 3, 2016

Related U.S. Application Data
(60) Provisional application No. 62/380,544, filed on Aug. 29, 2016.

(51) Int. Cl. A61B 50/36; A61L 2/26

(52) U.S. Cl. CPC A61B 50/362; A61B 2050/3009; A61L 2/26

(57) ABSTRACT

Methods and apparatus for a modular sharps container system. A system includes a first half shell sharps container, the first half shell sharps container comprising a flat back panel including a first set of attachment features, and a second half shell sharps container, the second half shell sharps container comprising a flat back panel including a second set of attachment features, the first set of attachment features of the flat back panel of the first half shell sharps container detachably joined to the second set of attachment features of the flat back panel of the second half shell sharps container.

### CROSS-REFERENCE TO RELATED APPLICATIONS

This application claims priority to U.S. Provisional Application No. 62/380,544, filed on Aug. 29, 2016. The disclosure of the prior application is considered part of and is incorporated by reference in the disclosure of this application.

### BACKGROUND OF THE INVENTION

The present invention generally relates to medical waste containers, and more particularly to a modular sharps container system.

In general, medical waste must be safely collected and disposed of. Medical waste can include, for example, gloves, needles, lancets, syringes, broken glass, scalpels, culture slides, culture dishes, broken capillary tubes, broken rigid plastic, exposed ends of dental wires, stents, laboratory slides and cover slips contaminated with infectious agents. Such medical waste is typically placed in a “sharps container” or a “sharps bin.” Sharps bins enable the safe storage and disposal of all categories of sharps waste.

In general, a sharps bin is a puncture-resistant container with a wide rectangular opening for horizontal loading of medical waste, having the size and dimensions of a domestic waste basket.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

The present invention generally relates to medical waste containers, and more particularly to a modular sharps container system.

In an aspect, the invention features a sharps container including a curved front wall, a flat or curved top panel attached to an upper portion of the curved front wall, a flat or curved bottom panel attached to a lower portion of the curved front wall, the dimensions and shape of the top panel and the bottom panel matched to the dimensions of the curve of the curved front wall, and a flat back panel attached to a back portion of the curved front panel, the top panel and bottom panel, the flat back panel including one or more attachment features.

In another aspect, the invention features a system including a first half shell sharps container, the first half shell sharps container comprising a flat back panel including a first set of attachment features and a second half shell sharps container, the second half shell sharps container comprising a flat back panel including a second set of attachment features, the first set of attachment features of the flat back panel of the first half shell sharps container detachably joined to the second set of attachment features of the flat back panel of the second half shell sharps container.

In still another aspect, the invention features a method including providing a first half shell sharps container, the first half shell sharps container comprising a flat back panel including a first set of attachment features, providing a second half shell sharps container, the second half shell sharps container comprising a flat back panel including a second second of attachment features, and linking the first set of attachment features of the flat back panel of the first half shell sharps container to the second set of attachment features of the flat back panel of the second half shell sharps container.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

The invention will be more fully understood by reference to the detailed description, in conjunction with the following FIGs., wherein:

FIG. 1 is a block diagram of an exemplary modular sharps container system.

FIG. 2 is a block diagram of an exemplary half shell sharps container.

FIG. 3 is a top view of two exemplary half shell sharps containers joined together.

FIG. 4 is a flow diagram.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

In the description below, the term “or” is intended to mean an inclusive “or” rather than an exclusive “or.” That is, unless specified otherwise, or clear from context, “X employs A or B” is intended to mean any of the natural inclusive permutations. That is, if X employs A, X employs B, or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

A sharps bin in accordance with the present invention is configured as a half cylindrical receptacle with appropriate portal for introduction of a sharps for disposal. Sharps can include, but are limited to, needles (whether or not attached to a syringe or covered by a plastic guard), IV tubing with needle attached, glass Pasteur pipettes, disposable glass pipettes, glass slides and cover slips, scalpels, razor blades, and lancets, and broken glass and splintered plastic, when contaminated with blood or other potentially infectious material, for example.

The sharps bin is adapted to be affixed or mounted securely and temporarily to a wall via a molded fixture or some other fixation device located on or part of the sharps bin. Moreover, the sharps bin is configured to mate or join with a second sharps bin of similar design such that the overall shape and dimensions of the paired sharps bins enable the two joined “half shells” to be placed efficiently into a cylindrical sterilization system, such as an autoclave. In general, an autoclave is an apparatus that uses pressure and steam to reach and maintain a temperature that is too high for any microorganisms or their spores to live.

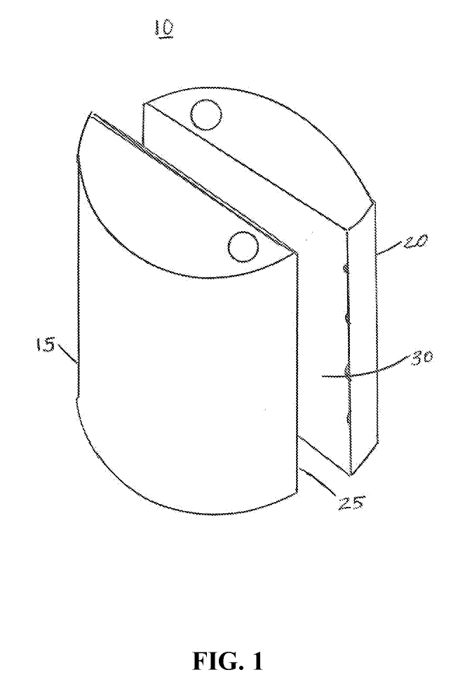

As shown in FIG. 1, an exemplary modular sharps container system 10 in accordance with the present invention includes a first sharps container 15 and a second sharps container 20 that may be detachably linked to each other. Each of the sharps containers 15, 20 is constructed in a half shell design that is puncture resistant and leak proof. More specifically, a back wall 25 of the first sharps container 15 mates with a back wall 30 of the second sharps container 20 to form a single modular structure. The overall shape and dimensions of the single modular structure formed by a joining of the first sharps container 15 to the second sharps container 20 enable the two joined “half shells” to be placed efficiently into a cylindrical sterilization system.

The mating of the back wall 25 and the back wall 30 may be effectuated with any suitable linking device, such as a molded feature, bracket, latch or the like. The selected molded feature, bracket or latch may be constructed of, for example, plastic or metal. More particularly, for example, the back wall 25 can include a first molded feature or a first set of molded features that is adapted to lock to a corresponding second molded feature or a second set of molded features on the back wall 30. In operation, the first molded feature and the second molded feature complement each to effectuate linking.

In implementations, the first molded feature on the back wall 25 and the second molded feature on the back wall 30 may be used to mount each of the first sharps container 15 and the second sharps container 20 separately to a wall for storage or during use individually, before both sharps containers 15, 20 are joined to each other. In other implementations, the back walls 25, 30 include additional mounting features to enable a wall mounting. These additional features can include supplemental molded features and/or mounting hardware, for example.

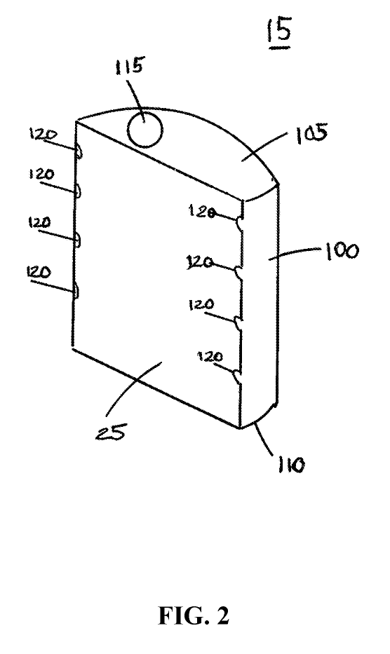

As shown in FIG. 2, each of the sharps containers, sharps container 15, for example, includes a generally rectangular half shell-like housing, formed of a generally puncture resistant and leak proof plastic material. More particularly, the sharps container 15 includes a front curved wall 100, a flat back wall or panel 25, a usually flat top panel 105 and a usually flat bottom panel 110. The shape of the top panel 105 and the bottom panel 110 are designed to match a curve of the front curved wall 100. The front curved wall 100, the flat back panel 25, the top panel 105 and the bottom panel 110 are preferably molded of a suitable puncture resistant and leak proof plastic.

The top panel 105 includes an opening 115 designed to receive syringes and the like for disposal. The opening 115 is designed in such a way as to be open-proof such that sharps cannot be retrieved easily once placed in the container 110.

The back panel 25 includes a first set of molded features 120. Each of the molded features 120 may enable the sharps container 15 to be mounted on a wall, or other features may be included that serve that function, as was described above. Moreover, in one specific embodiment, the first set of molded features 120 enable the sharps container 15 to be linked to corresponding second set of molded features on a back wall of second sharps container, such as the back panel 30 of second sharps container 20 in FIG. 1. As discussed above, an overall shape and dimensions of the single modular structure formed by the joining of the first sharps container 15 to the second sharps container 20 enable the two joined “half shells” to be placed efficiently into a cylindrical sterilization system.

In embodiments, the top panel 105 may also include a handle (not shown) to aid in handling the sharps container. In such a configuration, the handle may pivot outwardly for ease of grasping. In other embodiments, the sharps container side wall may be tapered such as may be required for certain molding technologies.

In normal operation, to dispose of a syringe, the syringe is inserted into the top opening 115. As soon as the first sharps container 15 is filled, the second sharps container 20 is filled in a likewise manner. Once both sharps containers 15, 20 are filled, they are dismounted from the wall and potentially linked to each other as described above, i.e., a first set of molded features on the first sharps container 15 is mated to a second set of molded features on the second sharps container 20. Once placed or linked together, the two “half shells” may be deposited efficiently into a cylindrical sterilization system for disposal, thereby maximizing the throughput of the sterilization system. The sterilization system may include, for example, an autoclave, a chemical treatment system, a gas vapor system, or other regulated waste processing machine.

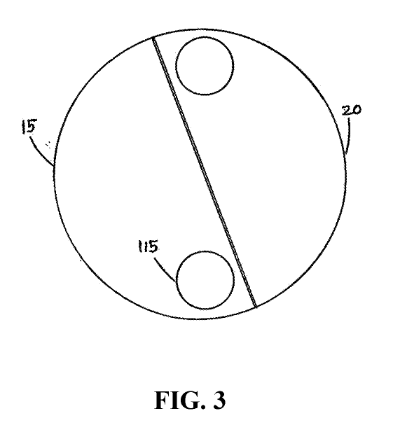

As shown in FIG. 3, a top view of sharps container 15 placed adjacent to or joined to sharps container 20 is illustrated and demonstrates the overall shape provided to enable placement into a cylindrical sterilization system. As mentioned previously, the back wall of sharps container 15 can be placed against the back wall of sharps container 20 for subsequent placement within the cylindrical sterilization system or the back wall of sharps container 15 can be joined to back wall of sharps container 20 using the first and second set of molded features, respectively, for subsequent placement within the cylindrical sterilization system.

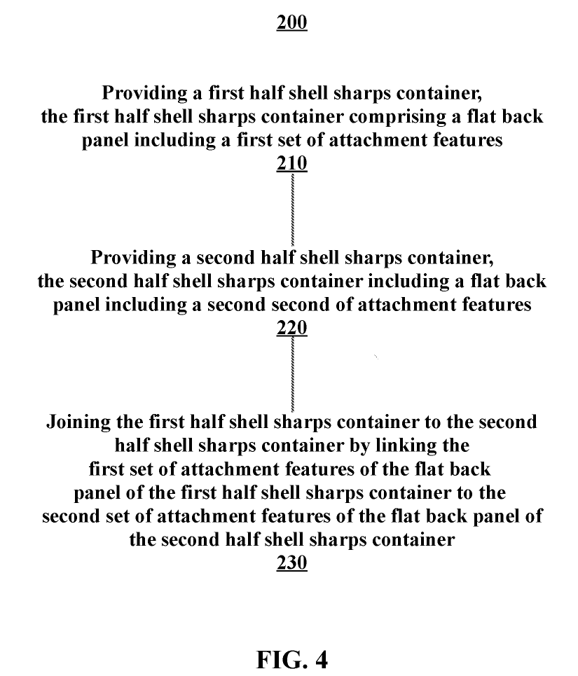

As shown in FIG. 4, a modular sharps container process 200 includes providing (210) a first half shell sharps container, the first half shell sharps container comprising a flat back panel including a first set of attachment features.

The modular sharps container process 200 includes providing (220) a second half shell sharps container, the second half shell sharps container comprising a flat back panel including a second set of attachment features.

The modular sharps container process 200 includes joining (230) the first half shell sharps container to the second half shell sharps container by linking the first set of attachment features of the flat back panel of the first half shell sharps container to the second set of attachment features of the flat back panel of the second half shell sharps container.

The overall dimensions of the joined first half shell sharps container and the second half shell sharps container enable placement into a cylindrical sterilization system.

While this invention has been particularly shown and described with references to preferred embodiments thereof, it will be understood by those skilled in the art that various changes in form and details may be made therein without departing from the spirit and scope of the present application as defined by the appended claims. Such variations are intended to be covered by the scope of this present application. As such, the foregoing description of embodiments of the present application is not intended to be limiting. Rather, any limitations to the invention are presented in the following claims.

1. (canceled)

2. The sharps container of claim 17, wherein said at least one attachment feature is selected from the group consisting of a molded fixture, a bracket and a latch.

3. The sharps container of claim 17, wherein the arcuate front wall, the top panel, the bottom panel, the back panel are constructed of a puncture resistant and leak proof material.

4. The sharps container of claim 17, wherein the top panel further comprises a handle.

5. The sharps container of claim 4 wherein the handle is a pivoting handle.

6. The sharps container of claim 17, wherein the arcuate front wall is tapered.

7-16. (canceled)

17. A sharps container for receiving and processing medical waste, said sharps container comprising:
a first bin portion, said first bin portion including an arcuate front wall, a top panel being attached to an upper portion of the front wall, a bottom panel being attached to a lower portion of the front wall, a back panel being attached to a back portion of the front panel;
wherein dimensions and shape of the top panel and the bottom panel are selected to correspond to dimensions and shape of the arcuate front wall;
wherein the arcuate front wall, top panel, bottom panel and back panel when so attached define an interior compartment in the first bin portion for receiving therein said medical waste;
wherein the first bin portion further includes a portal, the portal being configured and arranged to allow medical waste to be disposed in the interior compartment through the portal and to prevent removal of the medical waste from the interior compartment; and
at least one attachment feature being provided on the first bin portion.

18. The sharps container of claim 17, wherein the sharps container first bin portion is dimensioned and arranged such that the first bin portion can fit into one of a cylindrical sterilization system or a regulated waste processing machine for processing of the medical waste disposed in the interior compartment.

19. The sharps container of claim 18, wherein said one of a cylindrical sterilization system or a regulated waste processing machine comprises an autoclave, a chemical treatment system and a gas vapor system.

20. The sharps container of claim 17, further comprising a mounting mechanism that is adapted so said first bin portion can be secured temporarily to a building feature.

21. The sharps container of claim 17, wherein said medical waste includes needles (whether or not attached to a syringe or covered by a plastic guard), IV tubing with needle attached, glass Pasteur pipettes, disposable glass pipettes, glass slides and cover slips, scalpels, razor blades, and lancets, and broken glass and splintered plastic, including when contaminated with blood or other potentially infectious material.

22. The sharps container of claim 17, further comprising:
a second bin portion, said second bin portion including an arcuate front wall, a top panel being attached to an upper portion of the front wall, a bottom panel being attached to a lower portion of the front wall, a back panel being attached to a back portion of the front panel;
wherein dimensions and shape of the top panel and the bottom panel are selected to correspond to dimensions and shape of the arcuate front wall;
wherein the arcuate front wall, top panel, bottom panel and back panel when so attached define an interior compartment in the first bin portion for receiving therein said medical waste, the interior compartment of the second bin portion being separate from the interior compartment of the first bin portion;
wherein the first second portion further includes a portal, the portal being configured and arranged to allow medical waste to be disposed in the interior compartment through the portal and to prevent removal of the medical waste from the interior compartment; and
at least one attachment feature being provided on the first bin portion, said at east one attachment feature of each of the first and second portions being such as to allow the first bin portion and second bin portion to be removably attached to each other.

23. The sharps container of claim 22, wherein the sharps container first and second portions when secured to each other are dimensioned and arranged such that the combined first and second bin portions can fit into one of a cylindrical sterilization system or a regulated waste processing machine for processing of the medical waste disposed in the interior compartment

24. A sharps container for the storage and processing of medical waste, said sharps container comprising:
a first portion and a second portion wherein each of the first and second portions include an arcuate front wall, a top panel being attached to an upper portion of the front wall, a bottom panel being attached to a lower portion of the front wall, where dimensions and shape of the top panel and the bottom panel are selected to correspond to interior dimensions and shape of the front wall, and a back panel being attached to a back portion of the front panel;
wherein the front wall, top panel, bottom panel and back panel when so attached define an interior compartment in each of the first and second portions for receiving therein said sharps;
wherein each of the first and second sections each further include an opening therein, the opening being configured and arranged to allow a user to insert said sharp through the opening into the interior compartment of a respective first or second portion and also to prevent a person from later removing said sharp from the interior compartment; and
a plurality of attachment features being provided on each of the first and second portions, the plurality of attachment features being configured and arranged so that the first and second sections can be removable secured to each other;

25. The sharps container of claim 24, wherein the sharps container first and second portions when secured to each other are dimensioned and arranged such that the combined first and second portions can fit into one of a cylindrical sterilization system or a regulated waste processing machine for processing of
the medical waste disposed in the interior compartments.
